(19) United States
(12) Patent Application Publication
(10) Pub. No.: US 2017/0277826 A1
(43) Pub. Date: Sep. 28, 2017

(54) SYSTEM, METHOD AND SOFTWARE FOR ROBUST TRANSCRIPTOMIC DATA ANALYSIS

(71) Applicant: Insilico Medicine, Inc.

(72) Inventors: Ivan Ozerov, Baltimore, MD (US); Ksenia Lezhnina, Pyatigorsk (RU); Aleksandrs Zavoronkovs, London (GB); Alexander Aliper, Ramenskoye (RU); Artem Artemov, Moscow (RU); Nikolay Borisov, Krasnoznamensk (RU); Anton Buzdin, Istra (RU)

(21) Appl. No.: 15/469,604

(22) Filed: Mar. 27, 2017

Related U.S. Application Data
(60) Provisional application No. 62/313,752, filed on Mar. 27, 2016.

(51) Int. Cl. G06F 19/12; G06F 19/00; C40B 30/02; C12N 15/10; G06F 19/18; C12Q 1/68

(52) U.S. Cl. CPC G06F 19/12; G06F 19/18; C12Q 1/6886; C40B 30/02; C12N 15/1089; G06F 19/3437; C12Q 2600/158; C12Q 2600/136; C12Q 2600/106; G06F 19/20

(57) ABSTRACT

The present invention provides systems, methods and software for improving robustness of transcriptomic data analysis, the method including receiving control cell transcriptomic data (C) and cell transcriptomic data (S) under study for a gene, calculating a fold change ratio (fc) for the gene, repeating these steps for a plurality of genes, grouping co-expressed genes into modules, estimating gene importance factors based on a network topology, mapped from a plurality of the modules and obtaining a insilico Pathway Activation Network Decomposition Analysis (iPANDA) value, wherein the iPANDA value has a Pearson coefficient greater than a Pearson coefficient associated with another platform for manipulating the same data.

### FIELD

The present invention relates generally to systems and methods of analysis of gene signaling pathways, and more specifically to systems and methods for improving robustness of transcriptomic data analysis.

### BACKGROUND

In the twentieth century, enormous strides were made in combating infectious diseases, in their detection and drugs to treat them. The major problem in the medical world has thus shifted from treating acute diseases to treating chronic diseases. Over the last few decades, with the advent of genetic engineering, much research and funding has been invested in genomics and gene-based personalized medicine. A need has arisen to develop diagnostic tools for use in the characterization of personalized aspects of chronic diseases and diseases associated with aging.

Novel methods have been developed for screening for drugs that can minimize the difference between the various cellular or tissue states in a variety of tissues, while also taking into accounting for toxicity and adverse effect of the drug.

Intracellular signaling pathways regulate numerous processes involved in normal and pathological conditions including development, growth, aging and cancer. Many bioinformatics tools have been developed, which analyze signaling pathways.

The information relating to signaling pathway activation can be obtained from the massive proteomic or transcriptomic data. Although the proteomic level may be somewhat closer to the biological function of signaling pathway activation, the transcriptomic level of studies today is far more feasible in terms of performing experimental tests and analyzing the data.

US2008254497A provides a method of determining whether tumor cells or tissue is responsive to treatment with an ErbB pathway-specific drug. In accordance with the invention, measurements are made on such cells or tissues to determine values for total ErbB receptors of one or more types, ErbB receptor dimers of one or more types and their phosphorylation states, and/or one or more ErbB signaling pathway effector proteins and their phosphorylation states. These quantities, or a response index based on them, are positively or negatively correlated with cell or tissue responsiveness to treatment with an ErbB pathway-specific drug. In one aspect, such correlations are determined from a model of the mechanism of action of an ErbB pathway-specific drug on an ErbB pathway. Preferably, methods of the invention are implemented by using sets of binding compounds having releasable molecular tags that are specific for multiple components of one or more complexes formed in ErbB pathway activation. After binding, molecular tags are released and separated from the assay mixture for analysis.

U.S. Pat. No. 8,623,592 discloses methods for treating patients which methods comprise methods for predicting responses of cells, such as tumor cells, to treatment with therapeutic agents. These methods involve measuring, in a sample of the cells, levels of one or more components of a cellular network and then computing a Network Activation State (NAS) or a Network Inhibition State (NIS) for the cells using a computational model of the cellular network. The response of the cells to treatment is then predicted based on the NAS or NIS value that has been computed. The invention also comprises predictive methods for cellular responsiveness in which computation of a NAS or NIS value for the cells (e.g., tumor cells) is combined with use of a statistical classification algorithm. Biomarkers for predicting responsiveness to treatment with a therapeutic agent that targets a component within the ErbB signaling pathway are also provided.

The application of novel supervised learning algorithms to large-scale transcriptomic data has the potential to transform conventional approaches for disease classification, personalized medicine and the development of prognostic models; however, their use as a modality for clinical applications is hindered by several recognized challenges and limitation. First, one of the most relevant challenges in transcriptomic data analysis is the inherent complexity of gene network interactions, which remains a significant obstacle in building comprehensive predictive models from transcriptomic data. Moreover, high diversity of experimental platforms, difficulties to understand the obtained values and inconsistency of the data coming from the various types of equipment—may also lead to the incorrect interpretation of the underlying biological processes. Although a number of data normalization approaches have been proposed over the recent years (Walker, W. L. et al, 2008 and Shabalin et al, 2008)., it remains difficult to achieve robust results over a group of independent data sets even when they are obtained from the same profiling platform. This may be explained by a range of factors, such as wide heterogeneity among individuals on a population basis, variance in proliferation status and cell cycle stage in cells.

Despite these challenges, various transcriptomic data analysis algorithms have been developed by both academic and commercial settings, and numerous attempts have been made to apply these algorithms clinically, particularly, for predicting patient response to various anti-cancer therapies (Paik, 2004, Theurigen et al, (2006), Venkova (2015)).

Canonically, these approaches aimed to identify differentially expressed genes between groups of samples. Although this can lead to the identification of prospective genetic biomarkers and expression signature patterns of the process under study, it fails to capture subtle differences between samples that arise from dynamic interactions between genes at the systemic level. To circumvent this limitation a number of computational scoring platforms that can project gene expression data into a network of molecular signaling have been proposed for integrative pathway analysis (Braun et al, (2014)).

The major advantage of pathway-based methods is their capability to perform biologically relevant dimension reduction as a result of the analysis. However, despite significant advancements, current pathway-based methods are still imperfect in extrapolating the functional states of transcriptomes into the biological networks. Many popular pathway-based algorithms, such as Gene Set Enrichment Analysis (GSEA) and its extensions, rely solely on gene enrichment statistics, treating pathways as unstructured sets of genes (Subramanian, A. et al. (2005)). Another group, including Signaling Pathway Impact Analysis (SPIA), Topology GSA, and DEGraph, treats pathways as directed or undirected graphs representing networks of biochemical interactions at gene and protein levels (Tarca, A. L. et al. (2009), Jacob et al. (2010) Massa et al. (2010)).

The Oncofinder algorithm represents a halfway approach, where information about pathway topology is used to assign activation or repression roles of particular genes in the pathway and then estimate its overall activation (Buzdin, et al., Front Mol Biosci 1, (2014). Although very helpful, these approaches cannot overcome other above-mentioned limitations, posing a need for development of the new large-scale analytical methodologies that more accurately infer complex transcriptomic changes into the network of biologically relevant signaling axes.

There thus remains a need for systems and methods for robust transcriptomic data analysis. Prior art traditional methods for transcriptomic data analysis may not be sufficient in many cases. For example, breast cancer is the second most common cancer in the US, after skin cancer. Breast cancer is also the second leading cause of cancer death in women after lung cancer (Siegel, et al., Cancer J. Clin. (2015)). Hence, there is strong demand for the development of a new generation of highly robust transcriptomic data analysis methods.

### SUMMARY

It is an object of some aspects of the present invention to provide systems and methods, for improving transcriptomic data analysis.

The present invention provides novel methods for large-scale transcriptomic data analysis called insilico Pathway Activation Network Decomposition Analysis (iPANDA). The capabilities of this method are describe herein for multiple data sets containing data on Paclitaxel-based breast cancer treatment obtained from Gene Expression Omnibus (GEO). Breast cancer data was chosen for the analysis as one of the most challenging in several ways. Since breast tumor cells have large variance within the same cancer type, breast cancer nosology is one of the most difficult in terms of outcome and treatment response prediction. This is especially true for the most variable and hence the most lethal estrogen receptor negative (ERN) breast cancer types (both human epidermal growth factor receptor 2 (HER2) positive and negative).

There is thus provided according to an embodiment of the present invention, a method for improving robustness of transcriptomic data analysis, the method including;
a. receiving cell transcriptomic data of a control group (C) and cell transcriptomic data (S) of group under study for a gene;
b. calculating a fold change ratio (fc) for the gene;
c. repeating steps a and b for a plurality of genes;
d. grouping co-expressed genes into modules;
e. estimating gene importance factors based on a network topology, mapped from a plurality of the modules; and
f. obtaining an insilico Pathway Activation Network Decomposition Analysis (iPANDA) value, the iPANDA value having a Pearson coefficient greater than a Pearson coefficient associated with another platform for manipulating the control cell transcriptomic data and the cell transcriptomic data of group under study for the plurality of genes.

Additionally, according to an embodiment of the present invention, the method further includes;
g. determining a biological an insilico Pathway Activation Network Decomposition Analysis (iPANDA) associated with at least one the module.

Furthermore, according to an embodiment of the present invention, the method further includes;
h. providing a classifier for treatment response prediction of a drug to a disease, wherein the disease is selected from a cancer, a proliferative disease, an inflammatory disease and another disease or disorder.

Further, according to an embodiment of the present invention, the method further includes applying at least one statistical filtering test and a statistical threshold test to the fc values.

Yet further, according to an embodiment of the present invention, the method further includes;
i. obtaining proliferative bodily samples and healthy bodily samples from patients;
j. applying the drug to the patients; and
k. determining responder and non-responder patients to the drug.

Additionally, according to an embodiment of the present invention, the calculating step includes comparing gene expression in at least one of selected signaling pathways and metabolic pathways.

Moreover, according to an embodiment of the present invention, the selected signaling pathways are associated with the drug.

According to an additional embodiment of the present invention, the cancer is breast cancer.

Additionally, according to an embodiment of the present invention, the breast cancer is estrogen receptor negative (ERN) breast cancer.

Moreover, according to an embodiment of the present invention, the estrogen receptor negative (ERN) breast cancer is selected from HER2 positive and HER2 negative ERN breast cancer.

Furthermore, according to an embodiment of the present invention, the drug is Paclitaxel.

Importantly, according to an embodiment of the present invention, the Pearson coefficient is greater than 0.7 and less than 1.

Additionally, according to an embodiment of the present invention, the Pearson coefficient is greater than 0.75 and less than 0.95.

Further, according to an embodiment of the present invention, the Pearson coefficient is greater than 0.77 and less than 0.95.

Yet further, according to an embodiment of the present invention, the Pearson coefficient is greater than 0.79 and less than 0.94.

There is thus provided according to another embodiment of the present invention, a computer software product, the product configured for predicting drug efficacy for treating a disorder in a patient, the product including a computer-readable medium in which program instructions are stored, which instructions, when read by a computer, cause the computer to;
a. receive cell transcriptomic data of a control group (C) and cell transcriptomic data (S) of a group under study for a gene;
b. calculate a fold change ratio (fc) for the gene;
c. repeating steps a and b for a plurality of genes;
d. group co-expressed genes into modules;
e. estimate gene importance factors based on a network topology, mapped from a plurality of the modules; and
f. obtain an insilico Pathway Activation Network Decomposition Analysis (iPANDA) value, the iPANDA value having a Pearson coefficient greater than a Pearson coefficient associated with another platform for manipulating the control cell transcriptomic data and the cell transcriptomic data of group under study for the plurality of genes.

There is thus provided according to an additional embodiment of the present invention, a system for predicting drug efficacy for treating a disorder in a patient the system including;
a. a processor adapted to activate a computer-readable medium in which program instructions are stored, which instructions, when read by a computer, cause the processor to;
   i. receive cell transcriptomic data of a control group (C) and cell transcriptomic data (S) of group under study for a gene;
   ii. calculate a fold change ratio (fc) for the gene;
   iii. repeating steps a and b for a plurality of genes;
   iv. group co-expressed genes into modules;
   v. estimate gene importance factors based on a network topology, mapped from a plurality of the modules; and
   vi. obtain an insilico Pathway Activation Network Decomposition Analysis (iPANDA) value, the iPANDA value having a Pearson coefficient greater than a Pearson coefficient associated with another platform for manipulating the control cell transcriptomic data and the cell transcriptomic data of a group under study for the plurality of genes.
b. a memory for storing the data; and
c. a display for displaying data associated with a predictive indication of the patient.

The present invention will be more fully understood from the following detailed description of the preferred embodiments thereof, taken together with the drawings.

### BRIEF DESCRIPTION OF THE DRAWINGS

The invention will now be described in connection with certain preferred embodiments with reference to the following illustrative figures so that it may be more fully understood.

With specific reference now to the figures in detail, it is stressed that the particulars shown are by way of example and for purposes of illustrative discussion of the preferred embodiments of the present invention only and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1A is a simplified schematic illustration of a system for improving robustness of transcriptomic data analysis, in accordance with an embodiment of the present invention;

FIG. 1B is a simplified schematic illustration of a method for improving robustness of transcriptomic data analysis, in accordance with an embodiment of the present invention;

FIG. 2 shows Pearson sample-wise correlation coefficients between gene expression levels (differential genes only are used with group t-test p-value less than 0.05) obtained with Affymetrix and Agilent platforms for the same set of samples are shown at the bottom of the figure. Sample-wise correlations between corresponding pathway activation values calculated using iPANDA are shown at the top of the figure. Gene expression data was obtained from MicroArray Quality Control (MAQC) data set (GEO identifier GSE5350). Application of iPANDA leads to higher correlation between the data obtained using different experimental platforms for the same samples;

FIG. 3 shows ROC AUC values for 30 highest rated by AUC pathway markers of responders/non-responders to Paclitaxel-based ERN HER2P (left) and ERN HER2N (right) breast cancer treatment obtained using iPANDA. The saturation of the color denotes to corresponding AUC value. The same signaling pathways are found to be markers of responders/non-responders to Paclitaxel-based treatment for four independent data sets obtained from GEO. 21 and 14 out of 30 pathway markers for ERN HER2P and ERN HER2N breast cancer respectively demonstrate AUC values higher than 0.8 for all four data sets examined;

FIG. 4 shows Common marker pathway (CMP) index for responders/non-responders to Paclitaxel-based treatment of ERN HER2P and ERN HER2N breast cancer types. The index is calculated for four independent data sets obtained from GEO (GSE20194, GSE20271, GSE32646 and GSE50948) for each cancer type. Index demonstrates the robustness of the pathway marker lists between data sets. Independent application of the gene modules and topological coefficients did not improve the robustness of the algorithm for estimation of pathway activation; however, combined application resulted in significant improvement, in accordance with an embodiment of the present invention;

FIG. 5 shows the pairwise similarity plots between samples in pretreatment breast cancer data sets obtained from GEO. Subplots A and B show clustering of samples in four independent breast cancer data sets for ERN HER2P (A) and ERN HER2N (B) breast cancer types. The similarity was calculated using iPANDA values for 30 top ranked pathway markers with the highest AUC values shown in FIG. 3. Subplots C and E show the results of applying pathway markers found using training data sets (GSE20271, GSE32646 and GSE50948) to prediction of neoadjuvant treatment response in patients from MAQC-II data set (GSE20194) separately for ERN HER2P (C) and ERN HER2N (E) breast cancer types. Corresponding gene-level similarities for MAQC-II data set calculated using differentially expressed genes are shown in subplots D (ERN HER2P) and F (ERN HER2N). Pretreatment data with unknown receptor status (GSE22513) was successfully classified into responders and non-responders groups using previously obtained sets of pathway markers for ERN HER2N and ERN HER2P cancer types (G). Corresponding gene-level similarities for GSE22513 data set are given in subplot H, in accordance with an embodiment of the present invention. In all the figures similar reference numerals identify similar parts; and

FIG. 6 is a simplified schematic flowchart of a method for improving robustness of transcriptomic data analysis, in accordance with an embodiment of the present invention.

### DETAILED DESCRIPTION

In the detailed description, numerous specific details are set forth in order to provide a thorough understanding of the invention. However, it will be understood by those skilled in the art that these are specific embodiments and that the present invention may be practiced also in different ways that embody the characterizing features of the invention as described and claimed herein.

Some embodiments of the present invention are published in Ozerov, I. V., Lezhnina, K. V., Izumchenko, E., Artemov, A. V., Medintsev, S., Vanhaelen, Q., . . . & West, M. D. (2016). In silico Pathway Activation Network Decomposition Analysis (iPANDA) as a method for biomarker development. Nature Communications, 7, 13427, incorporated herein by reference in its entirety.

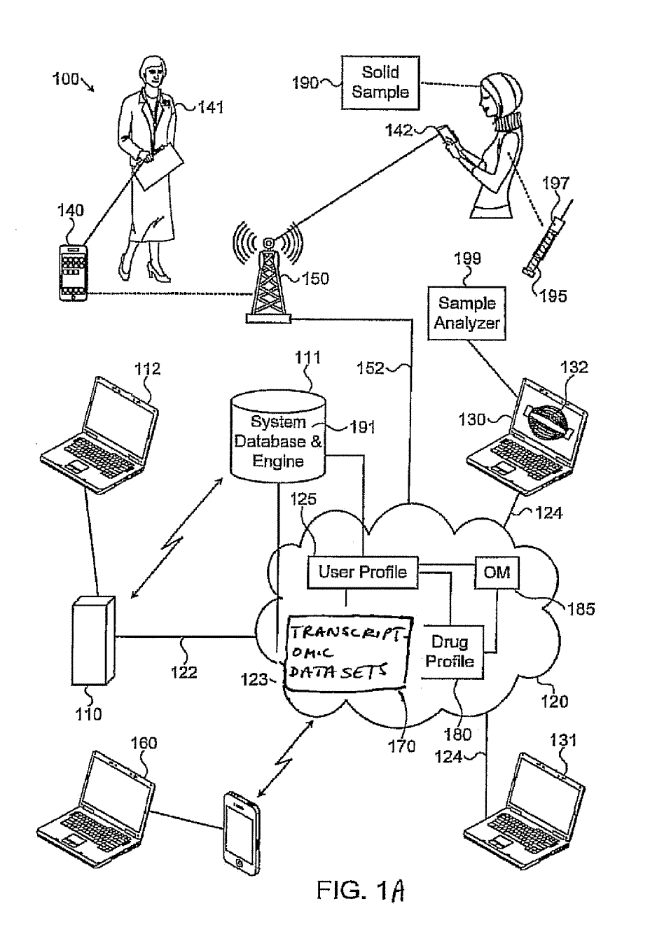

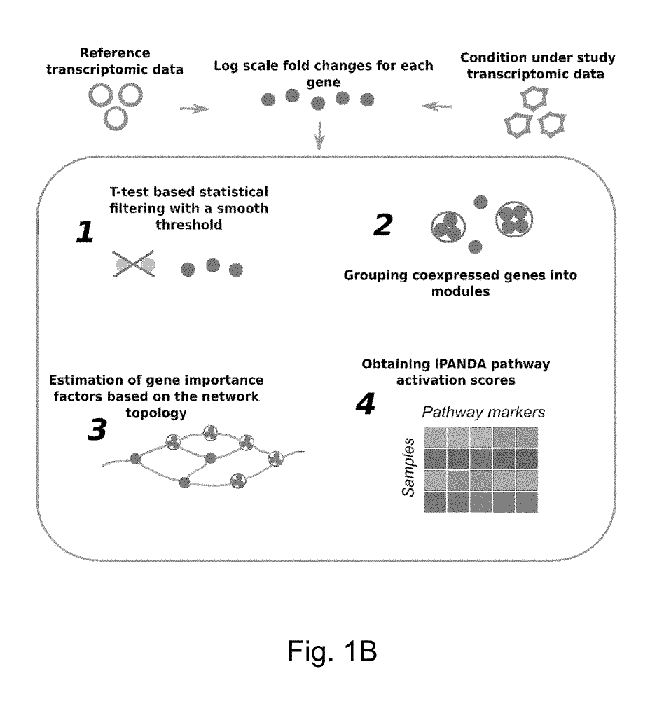

Reference is now made to FIG. 1A, which is a simplified schematic illustration of a system for improving robustness of transcriptomic data analysis, in accordance with an embodiment of the present invention.

System 100 typically includes a server utility 110, which may include one or a plurality of servers and one or more control computer terminals 112 for programming, trouble-shooting servicing and other functions. Server utility 110 includes a system engine 111 and database, 191. Database 191 comprises a user profile database 125, a pathway cloud database 123 and a drug profile database 180, as well as transcriptomic data sets 170.

The transcriptomic data sets may be stored, in part or wholly in the internet cloud 123, and/or in part or wholly in system database 111 and/or on various servers 110. The data sets may be public data sets or private subscription data sets. Some transcriptomic datasets are exemplified in the references listed hereinbelow, but should not be deemed as limiting to the invention.

Depending on the capabilities of a mobile device, system 100 may also be incorporated on a mobile device that synchronizes data with a cloud-based platform.

A medical professional, research personnel or patient assistant/helper/carer 141 is connected via his/her mobile device 140 to server utility 110. The patient, subject or child 143 is also connected via his/her mobile device 142 to server utility 110. In some cases, the subject may be a mammalian subject, such as a mouse, rat, hamster, monkey, cat or dog, used in research and development. In other cases, the subject may be a vertebrate subject, such as a frog, fish or lizard. The patient or child's is monitored using a sample analyzer 199. Sample analyzer 199, may be associated with one or more computers 130 and with server utility 110. Computer 130 and/or sample analyzer 199 may have software therein for predicting drug efficacy in a patient, as will be described in further details hereinbelow.

Typically, transcriptomic data sets 170 (FIG. 1A), generated by the software of the present invention, are stored locally and/or in cloud 120 and/or on server 110.

The sample analyzer may be constructed and configured to receive a solid (or liquid) sample 190, such as a biopsy, a hair sample or other solid sample, from patient 143, and/or a liquid sample 195, such as, but not limited to, urine, blood or saliva sample. The sample may be extracted by any suitable means, such as by a syringe 197.

The patient, subject or child 143 may be provided with a drug (not shown) by health professional/research/doctor 141.

System 100 further comprises an outputting module 185 for outputting data from the database via tweets, emails, voicemails and computer-generated spoken messages to the user, carers or doctors, via the Internet 120 (constituting a computer network), SMS, Instant Messaging, Fax through link 122.

Users, patients, health care professionals or customers 141, 143 may communicate with server 110 through a plurality of user computers 130, 131, or user devices 140, 142, which may be mainframe computers with terminals that permit individual to access a network, personal computers, portable computers, small hand-held computers and other, that are linked to the Internet 120 through a plurality of links 124. The Internet link of each of computers 130, 131, may be direct through a landline or a wireless line, or may be indirect, for example through an intranet that is linked through an appropriate server to the Internet. System 100 may also operate through communication protocols between computers over the Internet which technique is known to a person versed in the art and will not be elaborated herein.

Users may also communicate with the system through portable communication devices such as mobile phones 140, communicating with the Internet through a corresponding communication system (e.g. cellular system) 150 connectable to the Internet through link 152. As will readily be appreciated, this is a very simplified description, although the details should be clear to the artisan. Also, it should be noted that the invention is not limited to the user-associated communication devices—computers and portable and mobile communication devices—and a variety of others such as an interactive television system may also be used.

The system 100 also typically includes at least one call and/or user support and/or tele-health center 160. The service center typically provides both on-line and off-line services to users. The server system 110 is configured according to the invention to carry out the methods of the present invention described herein.

It should be understood that many variations to system 100 are envisaged, and this embodiment should not be construed as limiting. For example, a facsimile system or a phone device (wired telephone or mobile phone) may be designed to be connectable to a computer network (e.g. the Internet). Interactive televisions may be used for inputting and receiving data from the Internet. Future devices for communications via new communication networks are also deemed to be part of system 100. Memories may be on a physical server and/or in a virtual cloud.

A mobile computing device may also embody a non-synced or offline copy of memories, copies of pathway cloud data, transcriptomic data sets, user profiles database, drug profiles database and execute the system, engine locally.

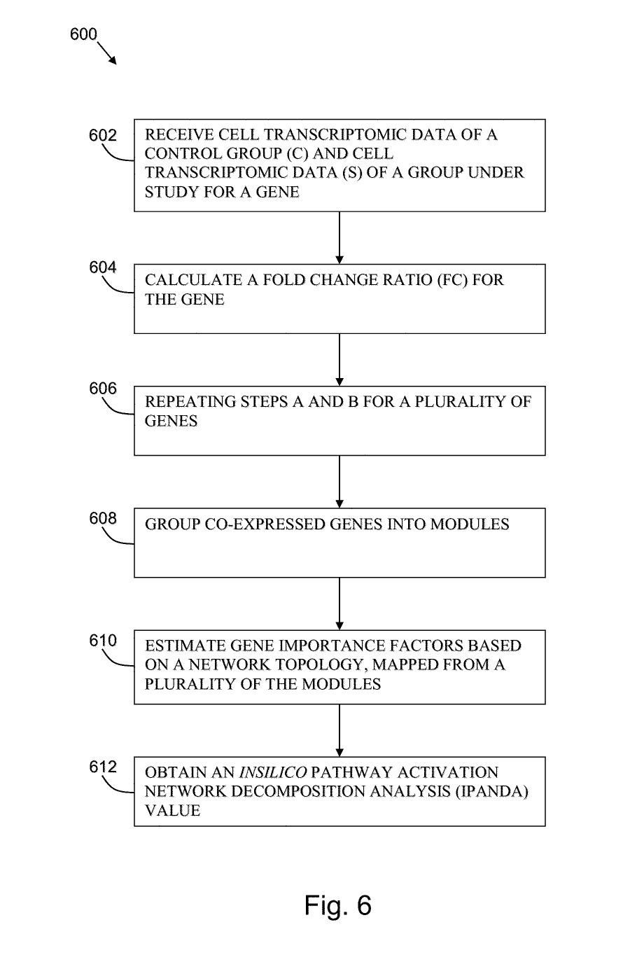

Turning to FIG. 6, there is seen a simplified schematic flowchart 600 of a method for improving robustness of transcriptomic data analysis, in accordance with an embodiment of the present invention.

As is described in further detail herein, the present invention provides a method for improving robustness of transcriptomic data analysis, per the steps of flowchart 600 of FIG. 6.

In a receiving cell transcriptomic data step 602, system 100 (FIG. 1A) is constructed and configured to receive cell transcriptomic data of a control group (C) and cell transcriptomic data (S) of group under study for a gene. The data may be obtained from transcriptomic datasets 170, available online or offline.

A computer or processor within system 100 is then operative to calculate a fold change ratio (fc) for the gene, in a calculating a fold change ratio (fc) for the gene step 604.

Steps 602 and 604 are then repeated in a repeating calculation step for n genes 606. This step may typically be performed by computer 112, server 110 or other processor within system 100.

In a grouping genes step 608, system 100 (FIG. 1A) is operative to group co-expressed genes into modules.

Thereafter, in an estimating gene importance factors step 610, the system is operative to estimate gene importance factors, based on a network topology, mapped from a plurality of the modules.

The method of the present invention yields an insilico Pathway Activation Network Decomposition Analysis (iPANDA) value, in an iPANDA outputting step 612, wherein the iPANDA value has a Pearson coefficient greater than a Pearson coefficient associated with another platform for manipulating the control cell transcriptomic data and the cell transcriptomic data of group under study for the plurality of genes.

### Results

An Overview of iPANDA Method

Fold changes between the gene expression levels in the samples under investigation (tumor samples) and an average expression level between a set of normal samples is used as input data for the iPANDA algorithm. Since some genes may have a stronger influence on pathway activation than others, the gene importance factor (GIF) has been introduced. Several approaches of gene importance hierarchy calculation have been proposed during the last few decades. All of these approaches aim to enrich pathway-based models with specific gene markers that are the most relevant for a given study. Some of them use detailed kinetic models of several particular metabolic networks to derive importance factors (Borisov et al. (2008)). In others, gene importance is derived from the statistical analysis of the gene expression data obtained for case and normal samples. Other approaches are based on the topological decomposition of the pathway maps and tend to give higher weight to the genes that occupy the central positions on the map (Gu et al. (2012)). Importantly, however, the measure of gene centrality strongly varies between algorithms. The novel approach integrates the different analytical concepts described above as it simultaneously exploits statistical and topological weights for gene importance estimation.

The smooth threshold, defined hereinbelow, based on the p-values from a t-test performed on groups of normal and tumor samples is applied to the gene expression values. The smooth threshold is defined as a continuous function of p-value ranging from 0 to 1. The statistical weights are also derived during this procedure. The topological weights for genes are obtained during the pathway map decomposition procedure. The topological weight of each gene is proportional to the number of independent paths through the pathway gene network represented as a directed graph.

It is well known that many genes often exhibit considerable correlation in expression levels (Okamura et al. (2015)). However, most algorithms for pathway analysis treat gene expression levels as independent variables. Such an approach is particularly inappropriate when topology-based coefficients are applied. In fact, if a set of coexpressed genes has correlated expression levels and hence fold change values in the data set, there is no significant dependence of pathway activation values on how the topology weights are distributed over these genes. Thus, the computation of topological coefficients for a set of coexpressed genes is inefficient, unless a group of coexpressed genes is being considered as a single unit. For this reason, gene modules reflecting the coexpression of genes were introduced in the iPANDA algorithm. The wide database of gene coexpression in human samples, COEXPRESdb (Okamura et al. (2015), and the database of the downstream genes controlled by various transcriptional factors (Zheng et al. (2008)) are utilized for grouping genes into modules. Thus the topological coefficients are estimated for each gene module as a whole rather than for individual genes inside the module.

The contribution of gene units (including gene modules and individual genes) to pathway activation is computed as a product of their fold changes in logarithmic scale, topological and statistical weights. Then the contributions are multiplied by a discrete coefficient which equals to +1 or −1 in the case of pathway activation or suppression by the particular unit respectively. Finally the activation scores, referred to as iPANDA values, are obtained as a linear combination of the scores calculated for gene units that contribute to the pathway activation/suppression. Therefore, the iPANDA values are signed scores showing the intensity and direction of pathway activation (see Methods section for details).

Pathway Quality Metrics

Unfortunately, there is no standardized pipeline for the benchmarking of transcriptomic data analysis algorithms. We aim to generalize the approaches for pathway-based algorithm testing and reveal the common features of reliable pathway-based expression data analysis. We term these features “pathway analysis quality hallmarks”. Efficient methods for pathway-based transcriptomic data analysis should be capable to perform a significant noise reduction in the input data and aggregate output data as a small number of highly informative features (pathway markers). Scalability (the ability to process pathways with small or large numbers of genes similarly), is another critical aspect that should be considered when designing a reliable pathway analysis approach, since pathway activation values for pathways of different sizes should be equally credible. The list of pathway markers identified should be relevant to the specific phenotype or medical condition, and robust over multiple data sets related to the process or biological state under investigation. The calculation time should be reasonable to allow high-throughput screening of large transcriptomic data sets. To address the iPANDA algorithm in respect to these hallmarks and to fully assess its true potential and limitations, we have directly compared the results obtained by iPANDA using the breast cancer and MAQC datasets with two other widely used third-party viable alternatives, GSEA (Subramanian, A. et al. (2005)), SPIA (Tarca et al (2009)).

iPANDA is an Effective Tool for Noise Reduction in Transcriptomic Data

The first important issue that should be addressed by any suitable transcriptomic data analysis algorithm is the ability to reduce noise while retaining the biologically relevant information of the results. Since pathway-based analysis algorithms are considered dimension reduction techniques, the pathway activation scores should represent collective variables describing only biologically significant changes in the gene expression profile.

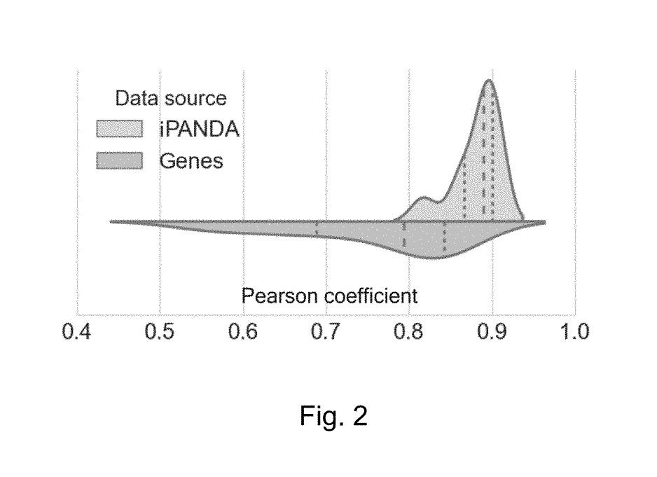

In order to estimate the ability of the iPANDA algorithm to perform noise reduction while preserving biologically relevant features, an analysis was performed using the well-known MicroArray Quality Control (MAQC) data set (GEO identifier GSE5350) (Shippy et al, (2006)). It contains data for the same cell samples processed using various transcriptome profiling platforms. A satisfactory pathway or network analysis algorithm should reduce the noise level and demonstrate a higher degree of similarity between the samples in comparison to the similarity calculated using gene set data. To estimate gene level similarity, only fold changes for differentially expressed genes (t-test p-value<0.05) were utilized. Pearson correlation was chosen as a metric to measure the similarity between samples. Sample-wise correlation coefficients were obtained for the same samples profiled on Affymetrix and Agilent platforms. Similar procedure was performed using pathway activation values (iPANDA values). The results acquired for the set of samples from MAQC data are shown in FIG. 2. Notably, the similarity calculated using pathway activation values generated by the iPANDA algorithm significantly exceeds the one calculated using fold changes for the differentially expressed genes (mean sample-wise correlation was over 0.88 and 0.79, respectively). For further validation the algorithm's noise reduction efficacy was compared with that of other routinely used methods for transcriptome-based pathways analysis, such as SPIA and GSEA. The mean sample-wise correlation between platforms was 0.88 for iPANDA compared to 0.53 for GSEA and 0.84 for SPIA. Furthermore, the sample-wise correlation distribution obtained using iPANDA values is narrowed to a range of 0.79 to 0.94, compared to 0.60-0.92 and −0.08-0.80 for SPIA and GSEA respectively. Taken together, iPANDA demonstrates better performance for the noise reduction test in comparison to other pathway analysis approaches suggesting that its credibility as a powerful tool for noise reduction in transcriptomic data analysis.

Biomarker Identification and Relevance

As a next step, the iPANDA ability to identify biomarkers (or pathway markers) of the process or phenotype under investigation was addressed. One of the commonly used methods to assess the capability of transcriptomic pathway markers to distinguish between two groups of samples (e.g. resistance and sensitivity to treatment) is to measure their ROC AUC values. The capacity to generate a high number of biomarkers with high AUC values is a major requirement for any prospective transcriptomic data analysis algorithm.

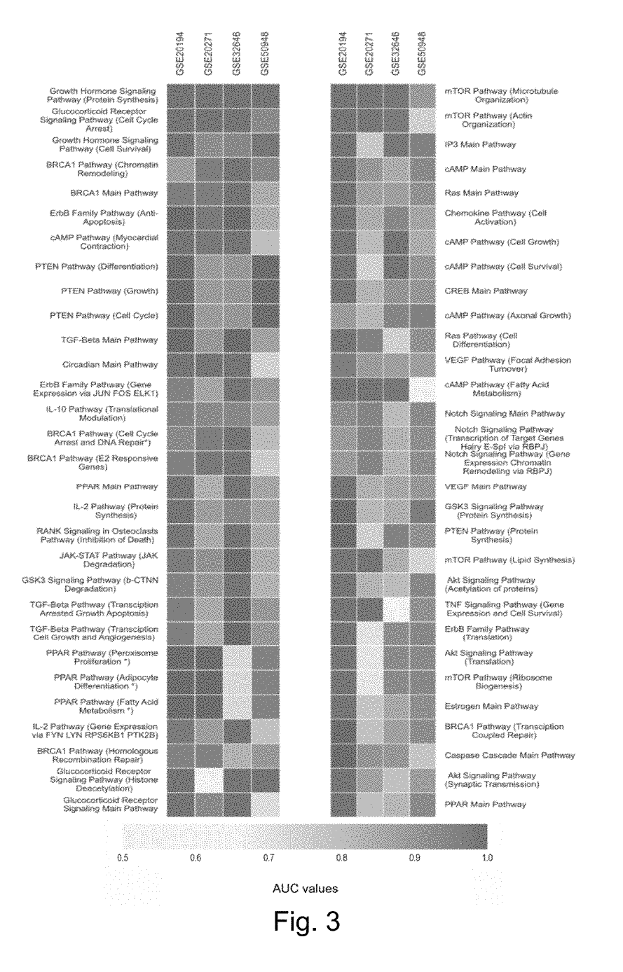

To estimate the capability of the methods to produce potential biomarkers, several gene expression data sets from breast cancer patients were chosen with measured response to Paclitaxel-based treatment. iPANDA algorithm was applied to obtain pathway activation scores for each sample and lists of the top 30 Paclitaxel treatment sensitivity pathway markers obtained for the ERN HER2P and ERN HER2N breast cancer types are given in FIG. 3. Signaling pathways were ranked by their average AUC values over four independent data sets. Pathways like ErbB, PTEN, BRCA1, PPAR, TGF-beta and RAS, which were previously reported to trigger Paclitaxel treatment response, can be found in these lists (Harari et al. (2000), Gilmore et al. (2004), Chen et al., (2012) and (Bhola et al., (2013)). Although the iPANDA-generated pathway marker lists obtained within data on the same cancer type have noticeable intersection, the lists of markers differ significantly between cancer types. This complies with the observation that the mechanisms of Paclitaxel treatment resistance are sensitive to cancer type (Baselga et al., (1997), Nelson (2000)).

Pathways with various numbers of member genes ranging from less than 10 members (VEGF pathway adhesion turnover) to over 400 (Ras main pathway) can be found on the lists. This illustrates that iPANDA algorithm treats smaller pathways in the same way as larger ones, hence demonstrates the scalability hallmark of valid pathway analysis methods.

iPANDA Produces Highly Robust Set of Biomarkers

One of the most important shortcomings of modern pathway analysis approaches is their inability to produce consistent results for different data sets obtained independently for the same biological case. iPANDA algorithm applied to the breast cancer data is shown to overcome this flaw and produce highly-consistent set of pathway markers across the data sets examined. In particular, the iPANDA values for 26 and 14 pathways for ERN HER2P and ERN HER2N breast cancer types, respectively, can be utilized as Paclitaxel response classifiers with AUC values higher than 0.8 for all four data sets. On the contrary, all of the third-party algorithms tested (including GSEA, SPIA) are not able to obtain even a single pathway marker with the same AUC threshold equal to 0.8 common to all four data sets examined for both cancer types. These findings suggest that the iPANDA is an extremely favorable choice for biologically relevant pathway marker development compared to the other pathway-based algorithms.

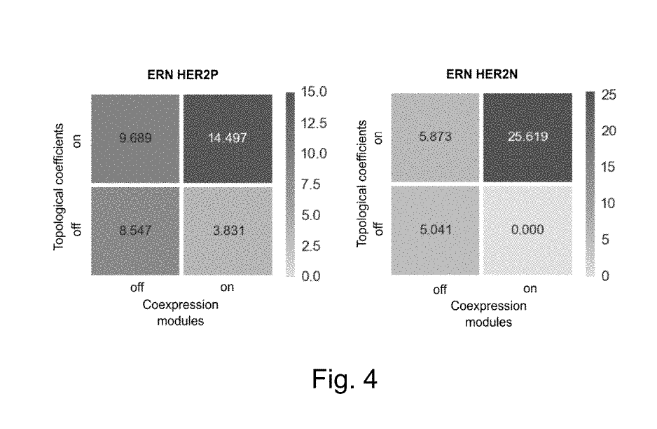

The Common Marker Pathway (CMP) index (see Methods section for details) was applied to Paclitaxel treatment response data for both ERN HER2P and ERN HER2N breast cancer types in order to estimate the robustness of the biomarker lists. Pathway marker lists obtained for four independent data sets were analyzed. The calculation of pathway activation scores was performed using the original iPANDA algorithm and its variants with disabled gene grouping and/or topological weights. The results of the calculation are shown in FIG. 4. The “off” state of topology coefficients means that they are equal to 1 for all genes during the calculation. Also the “off” state for the gene grouping means that all the genes are treated as individual genes. The application of the gene modules without topology-based coefficients reduces the robustness of the algorithm as well as the overall number of common pathway markers between data sets. Turning on the topology-based coefficients just slightly increases the robustness of the algorithm, whereas using topology with gene modules simultaneously improves this parameter drastically for both cancer types. This result implies that the combined implementation of the gene modules along with the topology-based coefficients serves as an effective way of noise reduction in gene expression data and allows one to obtain stable pathway activation scores for a set of independent data.

The Application of iPANDA Biomarkers as Classifiers for Treatment Response Prediction

The pathway activation scores obtained using iPANDA were applied to the identification of Paclitaxel chemotherapy sensitivity in breast cancer. The normalized iPANDA values were calculated for all samples in four data sets and merged for the clustering procedure. The major part of the activation scores splits well for the responders and non-responders groups respectively. Hence the iPANDA values can be considered promising classifiers for Paclitaxel treatment response prediction. On the other hand, the scores obtained using other pathway-based algorithms overlap significantly between groups for the majority of the pathway markers.

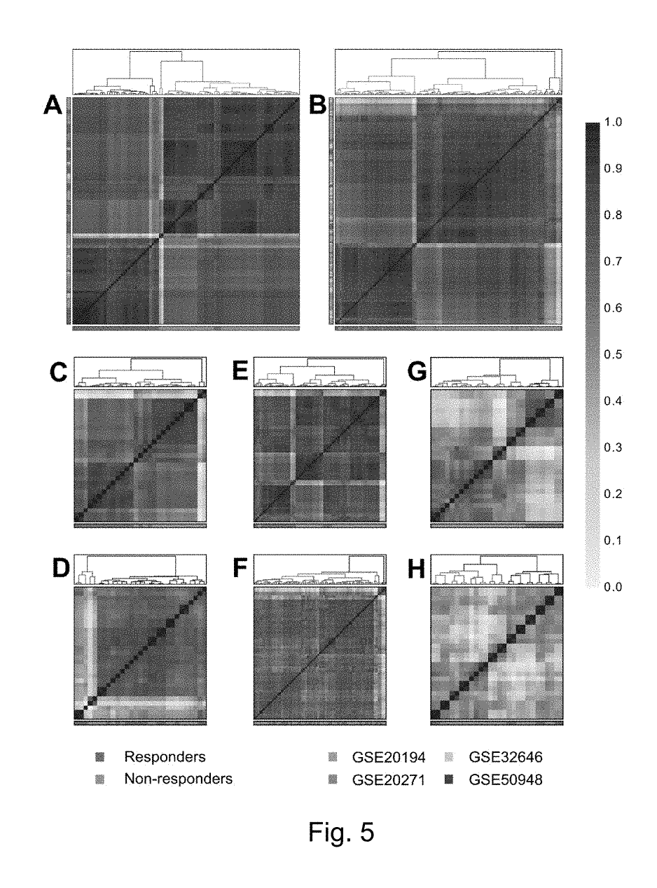

The pairwise distance matrices between samples were calculated using the 30 top pathway markers, ranked by their average AUC values for ERN HER2P and ERN HER2N breast cancer data (see Methods section). In order to classify the samples as responders or non-responders, hierarchical clustering using Ward linkage was performed on the distance matrices (FIG. 5). Distinct clusters containing mostly the samples from responders or non-responders groups, respectively, were obtained for both cancer types. The ratios of false positive (type I error) and false negative (type II error) predictions of Paclitaxel treatment response are given in Table 1. The similar clustering procedure using biomarkers and pathway activation (enrichment) scores obtained using other algorithms failed to distinguish between the responders and non-responders groups.

TABLE 1
False positive and false negative ratios for Paclitaxel
treatment response prediction.

| | Data | False | False |
|---|---|---|---|
| | utilized for | positive | negative |
| Cancer type | prediction | ratio | ratio |
| ERN HER2P | GSE20194 | 0.000 | 0.051 |
| | GSE20271, | | |
| | GSE32646, | | |
| | GSE50948 | | |
| | MAQC-II | 0.000 | 0.125 |
| | (GSE20194) | | |
| ERN HER2N | GSE20194, | 0.115 | 0.057 |
| | GSE20271, | | |
| | GSE32646, | | |
| | GSE50948 | | |
| | MAQC-II | 0.111 | 0.120 |
| | (GSE20194) | | |

As it can be seen in FIGS. 5(A,B), the clustering performed using normalized iPANDA values distinguishes Paclitaxel treatment responders from non-responders. On the other hand, clustering becomes insensitive to the distinctions between data sets and the samples from different data sets are mixed upon the hierarchical tree for both cancer types under investigation. The use of 10 top-ranked pathway markers for Paclitaxel response prediction in ERN HER2P patients gives 100% accuracy. ERN HER2N breast cancer and especially its triple negative subclass (also progesterone receptor negative) is known to have the most diverse phenotype (Podo et al. (2010)). Therefore searching for an effective therapy and prediction of the therapy outcome for this type of breast cancer is a challenging task. Nevertheless, the application of iPANDA values as classifiers for Paclitaxel treatment response prediction in ERN HER2N breast cancer showed approximately 90% accuracy. Thus such a result appears to be a significant success.

Besides, taxol-based neoadjuvant therapy response prediction was performed using another pretreatment data set (GSE22513) with unspecified receptor status, but known treatment outcome. A set of 30 pathway markers which consisted of 15 top pathway markers previously obtained for ERN HER2P cancer type and 15 top pathway markers obtained for ERN HER2N cancer type. All 28 samples (8 responders and 20 non-responders) from the data set were correctly separated into two groups. The results show that patient classification according to their potential treatment response can be successful even in case when receptor status is unknown. These finding suggest that despite the differences between breast cancer types, both ERN HER2P and ERN HER2N cancer hold common features which can define treatment response sensitivity. However, stratification of data from four previously discussed breast cancer data sets was impossible without accounting receptor status (data not shown).

In order to estimate the cross-study validity of iPANDA pathway marker application to treatment response prediction in breast cancer, prediction using separate training and test data sets was performed. Training set consisted of three independent pretreatment data sets (GSE20271, GSE32646 and GSE50948) with known treatment outcome and receptor status. Training set was used for obtaining list of 30 pathway markers with highest AUC values. MAQC-II breast cancer data set (GSE20194) was used as a test set. ERN HER2N and ERN HER2P samples were extracted using information about MAQC-II endpoint D (HER2 and ER receptor status). The samples were classified then, separately for each cancer type based on their iPANDA scores for pathway markers obtained from the training set. Results were compared to the actual data about treatment outcome (endpoint E in MAQC-II data). Results of MAQC-II data classification are shown in FIG. 5(C,F). False negative and false positive rates of predictions are given in Table 1. FIG. 5(D,F) shows corresponding similarity plots obtained using gene-level data (differentially expressed genes with group t-test p-value <0.05) for ERN HER2P and ERN HER2N cancer types in MAQC-II data set. It can be seen that in contrast to stratification based on iPANDA pathway markers, gene-level based classification fails to produce clusters corresponding to Paclitaxel treatment sensitivity. This data confirms that highly robust sets of pathway markers produced by iPANDA provide potential for wider applications of transcriptomic data analysis especially in case when cross-study comparison is inevitable.

Further Perspectives of iPANDA Application

Application of the pathway activation measurement implemented in iPANDA leads to significant noise reduction in the input data and hence enhances the ability to produce highly consistent sets of biologically relevant biomarkers acquired on multiple transcriptomic data sets. Another advantage of the approach presented is the high speed of the computation. The gene grouping and topological weights are the most demanding parts of the algorithm from the perspective of computational resources. Luckily these parts can be precalculated only once prior to the actual calculations using transcriptomic data. The calculation time for a single sample processing equals approximately 1.4 seconds on the Intel (R) Core i3-3217U 1.8 GHz CPU (compared to 1.2 seconds for PAS, 10 seconds for GSEA and 10 minutes 08 seconds for SPIA). Thus iPANDA can be an efficient tool for high-throughput biomarker screening of large transcriptomic data sets.

The use of merely microarray data for pathway activation analysis has well-known limitations, as it cannot address individual variations in the gene sequence and consequently in the activity of its product. For example, a gene can have a mutation that reduces activity of its product but elevates its expression level, through a negative feedback loop. Thus the elevated expression of the gene does not necessarily correspond to the increase in the activity of its product. Although the iPANDA algorithm was initially designed for microarray data analysis it can also be easily applied to the data derived from Genome-Wide Association Studies (GWAS). In order to do so, GWAS data can be converted to a form amenable for the iPANDA algorithm. Single point mutations (SNPs) are assigned to the genes based on their proximity to the reading frames. Then each SNP is given a weight derived from a GWAS data statistical analysis (Torkamani et al. (2008). Simultaneous use of the GWAS data along with microarray data may improve the predictions made using the iPANDA method.

Recently, several successful studies on microarray data analysis using various deep learning approaches on gene-level data have surfaced (Hira et al. (2015). From an experimental point of view, gene regulatory networks are controlled via activation or inhibition of a specific set of signaling pathways (set is dependent on the type of protocol and outcome expected). Thus, using the iPANDA signaling pathway activation scores as input for deep learning methods could bring results closer to experimental reality and make them more interpretable to bench biologists. One of the most difficult steps of multilayer perceptron training is the dimension reduction and feature selection procedures which aim to generate the appropriate input for further learning (Ibrahim et al. (2014). Signaling pathway activation scoring using iPANDA will likely help reduce the dimensionality of expression data without losing biological relevance and may be used as an input to deep learning methods. Using iPANDA values as an input data seems to be a particularly promising approach to obtaining reproducible results when analyzing transcriptomic data from multiple sources.

Methods.

Transcriptomic Data.

From the GEO database, four data sets were downloaded containing human gene expression data related to breast cancer with Paclitaxel treatment and two data sets related to normal breast tissue which were used as a reference (Table 2).

TABLE 2
Transcriptomic data sets on Paclitaxel breast cancer treatment
utilized in the study.

| Breast cancer tissue | | |
|---|---|---|
| data sets (total number | Normal tissue data sets | Affymetrix |
| of samples) | (number of samples used) | platform |
| GSE20194 (278) | GSE9574 (15) | GPL96 |
| GSE20271 (178) | GSE9574 (15) | GPL96 |
| GSE32646 (115) | GSE42568 (10) | GPL570 |
| GSE50948 (156) | GSE42568 (10) | GPL570 |
| GSE22513 (28) | GSE42568 (10) | GPL570 |

The breast cancer and normal data from different data sets was preprocessed using GCRMA algorithm (Wu et al. (2005)) and summarized using updated chip definition files from Brainarray repository (Version 18) (Dai et al. (2005)) for each data set independently.

Estrogen receptor negative (ERN) breast cancer samples were stratified by the HER2 receptor status: human epidermal growth factor receptor-2 positive (HER2P) and human epidermal growth factor receptor-2 negative (HER2N). Only samples profiled before any treatment were analyzed. In this analysis, the samples from patients who afterwards underwent Paclitaxel (Taxol) treatment were included and showed any definite therapy outcome (response or non-response) (Table 3). Also GSE22513 breast cancer data set with known Paclitaxel treatment outcome and unspecified receptor status was utilized. It contains samples from 20 non-responders and 8 responders patients.

TABLE 3
The number of ERN HER2P and ERN HER2N breast cancer samples from
patients treated with Paclitaxel with a distinct outcome.

| | Number of | Number of | Number of | Number of |
|---|---|---|---|---|
| | responders | non-responders | responders | non-responders |
| GEO ID | ERN HER2P | ERN HER2P | ERN HER2N | ERN HER2N |
| GSE20194 | 15 | 14 | 27 | 46 |
| GSE20271 | 6 | 3 | 10 | 25 |
| GSE32646 | 9 | 9 | 10 | 16 |
| GSE50948 | 9 | 30 | 6 | 11 |
| Total | 39 | 56 | 53 | 88 |

The MicroArray Quality Control (MAQC) data set (GEO identifier 5350) was obtained from the GEO database. The raw data for 60 samples from Affymetrix platform was preprocessed using GCRMA algorithm (Wu, et al., J. Comput. Biol. (2005) and summarized using updated chip definition files from Brainarray repository (Version 18) (Dai, et al., Nucleic Acids Res., (2005) for each data set independently. The preprocessed data for 60 samples from Agilent platform was taken as provided by authors. These samples represent 4 different groups: A=Stratagene Universal Human Reference RNA (UHRR, Catalog #740000), Sample B=Ambion Human Brain Reference RNA (HBRR, Catalog #6050), Sample C=Samples A and B mixed at 75%:25% ratio (A:B); and Sample D=Samples A and B mixed at 25%:75% ratio (A:B). Group A was used as a reference.

Pathway Database Overview

There are several widely used collections of signaling pathways including Kyoto Encyclopedia of Genes and Genomes (KEGG), QIAGEN and NCI Pathway Interaction Database. In this study, the collection of signaling pathways most strongly associated with various types of malignant transformation in human cells were used, obtained from the SABiosciences collection (www.sabiosciences.com/pathwaycentral.php). Using a cancer-specific pathway database ensures the presence of multiple pathway markers for the particular nosology of the breast cancer under investigation. The database contains a set of 374 signaling pathways which cover a total of 2294 unique genes. Each pathway contains an explicitly defined topology represented as a directed graph. Each node corresponds to a gene or a set of genes while edges describe biochemical interactions between genes in nodes and/or their products. All interactions are classified as activation or inhibition of downstream nodes. The pathway size ranges from about twenty to over six hundred genes in a single pathway.

Estimation of Pathway Activation

Our novel approach for large-scale transcriptomic data analysis accounts for the gene grouping into modules based on the precalculated gene coexpression data. Each gene module represents a set of genes which experience significant coordination in their expression levels and/or are regulated by the same expression factors (see grouping genes section below). Therefore the actual function for the calculation of the pathway p activation according to the proposed iPANDA algorithm consists of two terms. While the first one corresponds to the contribution of the individual genes, which are not members of any module, the second one takes into account the contribution of the gene modules. Therefore the final function for obtaining a iPANDA value for the activation of pathway p, which consists of the individual genes i and gene modules j, has the following analytical form:

$${iPANDA}_{p} = {\sum }_{i} {G}_{ix} + {\sum }_{i} {M}_{ip} \qquad ( 1 )$$

The contribution of the individual genes (Gᵢₚ) and the gene modules (Mⱼₚ) is computed as follows:

$${G}_{ip} = {w}_{i}^{s} \cdot {w}_{ip}^{T} \cdot {A}_{ip} \cdot lg ( {fc}_{i} ) \qquad ( 2 )$$

Here fcᵢ is the fold change of the expression level for the gene i in the sample under study to the normal level (average in a control group). As the expression levels are assumed to be logarithmically normally distributed and in order to convert the product over fold change values to sum, logarithmic fold changes are utilized in the final equation. Activation sign Aᵢₚ is a discrete coefficient showing the direction in which the particular gene affects the pathway given. It equals +1 if the product of the gene i has a positive contribution to the pathway activation and −1 if it has a negative contribution. The factors wᵢ^{S} and wᵢₚ^{T} are the statistical and topological weights of the gene i ranging from 0 to 1. The derivation procedure for these factors is described in detail in the subsequent sections. Since lg(fcᵢ) and Aᵢₚ values can be positive or negative, the iPANDA values for the pathways can also have different signs. Thus positive or negative iPANDA values correspond to pathway activation or inhibition respectively. The principal scheme of the iPANDA algorithm is shown in FIG. 1.

Obtaining Gene Importance Factors

In order to estimate the topological weight (wᵢₚ^{T}), all possible walks through the gene network are calculated on the directed graph associated with the pathway map. The nodes of the graph represent genes or gene modules, while the edges correspond to biochemical interactions. The nodes which have zero incoming edges are chosen as the starting points of the walks and those which have zero outgoing edges are chosen as the final points. Loops are forbidden during walks computation. The number of walks Nᵢₚ through the pathway p which include gene i is calculated for each gene. Then wᵢₚ^{T} is obtained as the ratio of Nᵢₚ to the maximum value of Nᵢₚ over all genes in the pathway:

$${w}_{ip}^{T} = \frac{{N}_{ip}}{max ( {N}_{jp} )} \qquad ( 4 )$$

The statistical weight depends on the p-values which are calculated from group t-test for case and normal sets of samples for each gene. The method called p-value thresholding is commonly used to filter out spurious genes which demonstrate no significant differences between sets. However, a major issue with the use of sharp threshold functions is that it can introduce an instability in filtered genes and as a consequence in pathway activation scores between the data sets. Additionally, the pathway activation values become sensitive to an arbitrary choice of the cutoff value. In order to address this issue, using a smooth threshold function is suggested. In the present study, the cosine function on logarithmic scale is utilized:

$${w}_{i}^{s} = \{ ( cos ( π \frac{log p - log {p}_{min}}{log {p}_{max} - log {p}_{min}} {{)}_{1 , p \leq {p}_{min}}}^{0 , p > {p}_{max}} + 1 ) / 2 , {p}_{min} < p \leq {p}_{max} \qquad ( 5 )$$

where pₘᵢₙ and pₘₐₓ are the high and low threshold values. In this study p-value thresholds equal to 10⁻⁷ and 10⁻¹ respectively. For the threshold values given over 58% of all genes pass high threshold and about 12% also pass low threshold for the breast cancer data under investigation. Hence over 45% of the genes in the data set receive intermediate wᵢ^{S} values. Therefore more stable results for pathway activation scores between data sets can be achieved using this approach.

Grouping Genes into Modules

To obtain the gene modules, two independent sources of data were utilized: human database of coexpressed genes COEXPRESdb (Okamura, et al., Nucleic Acids Res. (2015) and the database of the downstream genes controlled by human sequence-specific transcription factors (Zheng, et al., Bioinformatics, (2008)). The latter was simply intersected with the genes from the pathway database used, while correlation data from COEXPRESdb was clustered using Euclidean distance matrix. Distances were obtained according to the following equation:

rᵢⱼ=1−corrᵢⱼ (6)

where corrᵢⱼ is correlation between expression levels of genes i and j. DBScan and hierarchical clustering with an average linkage criteria were utilized to identify clusters. Only clusters with an average internal pairwise correlation higher than 0.3 were considered. Clusters obtained from the transcription factors database and coexpression database were recursively merged to remove duplicates. A pair of clusters was combined into one during the merging procedure if the intersection level between clusters had been higher than 0.7. As a result, a set of 169 gene modules which includes a total of 1021 unique genes was constructed.

Statistical Credibility of the iPANDA Values

The p-values for the iPANDA pathway activation scores are obtained using weighted Fisher's combined probability test. Thus the p-values (pₚ) are estimated according to the following equation:

$$ln ( {p}_{p} ) = \frac{{{\sum }_{i}}^{N} {w}_{i}^{S} \cdot {w}_{ip}^{T} \cdot ln ( {p}_{i} )}{\sqrt{{\sum }_{i}^{N} {( {w}_{i}^{S} \cdot {w}_{ip}^{T} )}^{2}}} \qquad ( 7 )$$

where i refers to the particular member (individual gene or gene module) of the pathway p, N is the number of pathway members, wᵢ^{T} and wₚ^{S} are the topological and statistical weights of the member i, pᵢ is the group t-test p-value for the member i. Since the p-values obtained are not used for further pathway marker scoring they are not normalized in any way and rely solely on the statistics for the genes which have non-zero statistical weights.

Algorithm Robustness Estimation

In order to quantitatively estimate the robustness of the algorithm between data sets, the Common Marker Pathway (CMP) index was introduced. The CMP index is a function of the number of pathways considered as markers that are common between data sets. It also depends on the quality of the treatment response prediction when these pathways are used as classifiers. The CMP index is defined as follows:

$$CMP = \frac{1}{n} {\sum }_{j = 1}^{n} {\sum }_{i} ln ( {N}_{i} ) \times ( {AUC}_{ij} - {AUC}_{R} ) \qquad ( 8 )$$

where n is the number of data sets under study, Nᵢ is the number of genes in the pathway i and AUCᵢⱼ is the value of the ROC area under curve which shows the quality of the separation between responders and non-responders to treatment when pathway i is used as classifier for the j-th data set. AUC_{R} is the AUC value for a random classifier and equals to 0.5. A pathway is considered as a marker if its AUC value is higher than 0.8. The ln(Nᵢ) term is included to increase the contribution of the larger pathways because they have a smaller probability to randomly get a high AUC value. The higher values of the CMP index correspond to the most robust prediction of pathway markers across the data sets under investigation, while zero value of CMP index corresponds to the empty intersection of the pathway marker lists obtained for the different data sets.

Clustering of Data Samples

In order to apply iPANDA to the Paclitaxel treatment response prediction over a several independent data sets, the pathway activation values were normalized to the Z-scores independently for each data set. The expected values used for the Z-scoring procedure were adjusted to the number of responders and non-responders in the data set under study. The pairwise distance matrix between samples utilized for further clustering was obtained using the Euclidean metric as follows:

$${D}_{i , j} = \sqrt{\frac{1}{N} \cdot {\sum }_{p}^{N} {( {iPANDA}_{ip} - {iPANDA}_{jp} )}^{2}} \qquad ( 9 )$$

Here Dᵢⱼ is the distance between samples i and j, N is the number of the pathway markers used for the distance calculation. iPANDAᵢₚ and iPANDAᵢₚ are the normalized iPANDA values for the pathway p for the samples i and j respectively. Normalization of iPANDA values to the Z-scores implies that all the considered pathway markers have an equal contribution to the distance obtained. All distances were converted into similarities (1−Dij) before the clustering procedure. Hierarchical clustering using Ward linkage was performed on the distance matrix to divide the samples into groups.

Performing Calculations Using Third Party Algorithms

In order to assess the results obtained with iPANDA algorithm from the perspective of modern advances in the pathway based transcriptomic data analysis three widely used third-party packages were selected for the comparison. GSEASPIA packages were chosen as the most commonly used.

A java GSEA package was downloaded from the GSEA official web site (www.broadinstitute.org/gsea/index.jsp). All the input data files were prepared according to the GSEA User guide available at software.broadinstitute.org/gsea/doc/GSEAUserGuideFrame.html. Only expression levels for differentially expressed genes (group t-test p-value less than 0.05) were used as the input. The pathway database was converted to the GSEA file format using the same package. All the calculations were run from the command line for each tumor sample versus all available normal samples for the particular data set. The parameter “Number of permutations” was set to 1000, “Collapse data set to gene symbols” was set to “false”, “Permutation type” was set to “gene_set”, “Enrichment statistics” was set to “weighted”, “Scoring scheme” was set to “weighted”, “Metric for ranking genes” was set to “Signal2Noise”, “Gene list sorting mode” was set to “real”, “Gene list ordering mode” was set to “descending”, “Collapsing mode for probe sets” was set to “max_probe”, “Normalization mode” was set to “meandiv”, “Randomization mode” was set to “no balance”. Normalized enrichment score (NES) values were extracted from GSEA report for further analysis.

SPIA R package was downloaded from Bioconductor Web site according to the instructions on the SPIA Bioconductor page (bioconductor.org/release/bioc/html/SPIA/html). The pathway database was converted to the SPIA file format using the same package. Fold changes between each tumor sample and the mean over the whole set of normal samples for the differentially expressed genes (group t-test p-value less than 0.05) were used as the input for the calculations. The total net accumulation perturbation (tA) values for each pathway were extracted from SPIA output for further analysis.

All the steps of further pathway analysis using GSEA, SPIA algorithms were similar to the ones used for the analysis performed using iPANDA algorithm.

It is to be understood that the invention is not limited in its application to the details set forth in the description contained herein or illustrated in the drawings. The invention is capable of other embodiments and of being practiced and carried out in various ways. Those skilled in the art will readily appreciate that various modifications and changes can be applied to the embodiments of the invention as hereinbefore described without departing from its scope, defined in and by the appended claims.

Unless specifically stated otherwise, as apparent from the following discussions, it is appreciated that throughout the specification discussions utilizing terms such as “processing”, “computing”, “calculating”, “determining”, “deriving”, “generating” or the like, refer to the action and/or processes of a computer or computing system, or processor or similar electronic computing device, that manipulate and/or transform data represented as physical, such as electronic, quantities within the computing system's registers and/or memories into other data, similarly represented as physical quantities within the computing system's memories, registers or other such information storage, transmission or display devices.

Embodiments of the present invention may use terms such as, processor, computer, apparatus, system, sub-system, module, unit, device (in single or plural form) for performing the operations herein. This may be specially constructed for the desired purposes, or it may comprise a general purpose computer selectively activated or reconfigured by a computer program stored in the computer. Such a computer program may be stored in a computer readable storage medium, such as, but not limited to, any type of disk including floppy disks, optical disks, CD-ROMs, Disk-on-Key, smart cards (e.g. SIM, chip cards, etc.), magnetic-optical disks, read-only memories (ROMs), random access memories (RAMs), electrically programmable read-only memories (EPROMs), electrically erasable and programmable read only memories (EEPROMs), magnetic or optical cards, or any other type of media suitable for storing electronic instructions capable of being conveyed via a computer system bus.

The processes/devices presented herein are not inherently related to any particular electronic component or other apparatus, unless specifically stated otherwise. Various general purpose components may be used in accordance with the teachings herein, or it may prove convenient to construct a more specialized apparatus to perform the desired method. The desired structure for a variety of these systems will appear from the description below. In addition, embodiments of the present invention are not described with reference to any particular programming language. It will be appreciated that a variety of programming languages may be used to implement the teachings of the inventions as described herein.

The references cited in the background teach many principles of computerized that are applicable to the present invention. Therefore the full contents of these publications are incorporated by reference herein where appropriate for teachings of additional or alternative details, features and/or technical background.

It is to be understood that the invention is not limited in its application to the details set forth in the description contained herein or illustrated in the drawings. The invention is capable of other embodiments and of being practiced and carried out in various ways. It should be noted that the invention is not bound by the specific algorithm of processing or specific structure. Those versed in the art will readily appreciate that the invention is, likewise, applicable to any other processing or presentation with equivalent and/or modified functionality which may be consolidated or divided in another manner.

It will also be understood that the invention further contemplates a machine-readable memory tangibly embodying a program of instructions executable by the machine for executing the method of the invention.

Those skilled in the art will readily appreciate that various modifications and changes can be applied to the embodiments of the invention as hereinbefore described without departing from its scope, defined in and by the appended claims.

### REFERENCES

Walker, W. L. et al. Empirical Bayes accommodation of batch-effects in microarray data using identical replicate reference samples: application to RNA expression profiling of blood from Duchenne muscular dystrophy patients. BMC Genomics 9, 494 (2008).
Shabalin, A. A., Tjelmeland, H., Fan, C., Perou, C. M. & Nobel, A. B. Merging two gene-expression studies via cross-platform normalization. Bioinformatics 24, 1154-1160 (2008).
Paik, S. et al. A multigene assay to predict recurrence of tamoxifen-treated, node-negative breast cancer. N. Engl. J. Med. 351, 2817-2826 (2004).
Thuerigen, O. et al. Gene expression signature predicting pathologic complete response with gemcitabine, epirubicin, and docetaxel in primary breast cancer. J. Clin. Oncol. 24, 1839-1845 (2006).
Venkova, L. et al. Combinatorial high-throughput experimental and bioinformatic approach identifies molecular pathways linked with the sensitivity to anticancer target drugs. Oncotarget 6, 27227-27238 (2015).
Braun, R. & Shah, S. Network Methods for Pathway Analysis of Genomic Data. arXiv [q-bio.QM] (2014). at <arxiv.org/abs/1411.1993>
Subramanian, A. et al. Gene set enrichment analysis: a knowledge-based approach for interpreting genome-wide expression profiles. Proc. Natl. Acad. Sci. U.S.A. 102, 15545-15550 (2005).
Tarca, A. L. et al. A novel signaling pathway impact analysis. Bioinformatics 25, 75-82 (2009).
Jacob, L., Neuvial, P. & Dudoit, S. Gains in Power from Structured Two-Sample Tests of Means on Graphs. arXiv [q-bio.QM] (2010). at <arxiv.org/abs/1009.5173>
Massa, M. S., Chiogna, M. & Romualdi, C. Gene set analysis exploiting the topology of a pathway. BMC Syst. Biol. 4, 121 (2010).
Buzdin, A. A. et al. The OncoFinder algorithm for minimizing the errors introduced by the high-throughput methods of transcriptome analysis. Front Mol Biosci 1, 8 (2014).
Edgar, R., Domrachev, M. & Lash, A. E. Gene Expression Omnibus: NCBI gene expression and hybridization array data repository. Nucleic Acids Res. 30, 207-210 (2002).
Rivenbark, A. G., O'Connor, S. M. & Coleman, W. B. Molecular and cellular heterogeneity in breast cancer: challenges for personalized medicine. Am. J. Pathol. 183, 1113-1124 (2013).
Putti, T. C. et al. Estrogen receptor-negative breast carcinomas: a review of morphology and immunophenotypical analysis. Mod. Pathol. 18, 26-35 (2005).
Siegel, R. L., Miller, K. D. & Jemal, A. Cancer statistics, 2015. CA Cancer J. Clin. 65, 5-29 (2015).
Borisov, N. M., Chistopolsky, A. S., Faeder, J. R. & Kholodenko, B. N. Domain-oriented reduction of rule-based network models. IET Syst. Biol. 2, 342-351 (2008).
Gu, Z., Liu, J., Cao, K., Zhang, J. & Wang, J. Centrality-based pathway enrichment: a systematic approach for finding significant pathways dominated by key genes. BMC Syst. Biol. 6, 56 (2012).
Okamura, Y. et al. COXPRESdb in 2015: coexpression database for animal species by
DNA-microarray and RNAseq-based expression data with multiple quality assessment systems. Nucleic Acids Res. 43, D82-6 (2015).
Zheng, G. et al. ITFP: an integrated platform of mammalian transcription factors. Bioinformatics 24, 2416-2417 (2008).
Shippy, R. et al. Using RNA sample titrations to assess microarray platform performance and normalization techniques. Nat. Biotechnol. 24, 1123-1131 (2006).
Harari, D. & Yarden, Y. Molecular mechanisms underlying ErbB2/HER2 action in breast cancer. Oncogene 19, 6102-6114 (2000).
Gilmore, P. M. et al. BRCA1 interacts with and is required for paclitaxel-induced activation of mitogen-activated protein kinase kinase kinase 3. Cancer Res. 64, 4148-4154 (2004).
Chen, Y. Z. et al. PPAR signaling pathway may be an important predictor of breast cancer response to neoadjuvant chemotherapy. Cancer Chemother. Pharmacol. 70, 637-644 (2012).
Bhola, N. E. et al. TGF-β inhibition enhances chemotherapy action against triple-negative breast cancer. J. Clin. Invest. 123, 1348-1358 (2013).
Blanchard, Z., Paul, B. T., Craft, B. & ElShamy, W. M. BRCA1-IRIS inactivation overcomes paclitaxel resistance in triple negative breast cancers. Breast Cancer Res. 17, 5 (2015).
Baselga, J., Seidman, A. D., Rosen, P. P. & Norton, L. HER2 overexpression and paclitaxel sensitivity in breast cancer: therapeutic implications. Oncology 11, 43-48 (1997).
Nelson, N. J. Can HER2 status predict response to cancer therapy? J. Natl. Cancer Inst. 92, 366-367 (2000).
Podo, F. et al. Triple-negative breast cancer: present challenges and new perspectives. Mol. Oncol. 4, 209-229 (2010).
Torkamani, A., Topol, E. J. & Schork, N. J. Pathway analysis of seven common diseases assessed by genome-wide association. Genomics 92, 265-272 (2008).
Hira, Z. M. & Gillies, D. F. A Review of Feature Selection and Feature Extraction Methods Applied on Microarray Data. Adv. Bioinformatics 2015, 198363 (2015).
Ibrahim, R., Yousri, N. A., Ismail, M. A. & El-Makky, N. M. Multi-level gene/MiRNA feature selection using deep belief nets and active learning. Conf. Proc. IEEE Eng. Med. Biol. Soc. 2014, 3957-3960 (2014).
Wu, Z. & Irizarry, R. A. Stochastic models inspired by hybridization theory for short oligonucleotide arrays. J. Comput. Biol. 12, 882-893 (2005).
Dai, M. et al. Evolving gene/transcript definitions significantly alter the interpretation of GeneChip data. Nucleic Acids Res. 33, e175 (2005).
Bauer-Mehren, A., Furlong, L. I., Sanz, F. (2009). Pathway databases and tools for their exploitation: benefits, current limitations and challenges. Mol Syst Biol, 5, article 290. doi: 10.1038/msb.2009.47.
Birtwistle, M. R., Hatakeyama, M., Yumoto, N., Ogunnaike, B. A. et al. (2007). Ligand-dependent responses of the ErbB signaling network: experimental and modeling analyses. Mol Syst Biol, 3, article 144. doi: 10.1038/msb4100188.
Borisov, N., Aksamitiene, E., Kiyatkin, A., Legewie, S. et al. (2009). Systems-level interactions between insulin-EGF networks amplify mitogenic signaling. Mol Syst Biol, 5, article 256, 2009. doi: 10.1038/msb.2009.19.
Borisov, N. M., Chistopolsky, A. S., Faeder, J. R., Kholodenko, B. N. (2008). Domain-oriented reduction of rule-based network models. IET Syst Biol, 2, 342-351. doi: 10.1049/iet-syb:20070081.
Borisov, N. M., Markevich, N. I., Hoek, J. B., Kholodenko, B. N. (2006). Trading the micro-world of combinatorial complexity for the macro-world of protein interaction domains. BioSystems, 83, 152-166. doi: 10.1016/j.biosystems.2005.03.006.
Borisov, N. M., Terekhanova, N. V., Aliper, A. M., Venkova, L. S., Smirnov, P. Y., Roumiantsev, S., . . . & Buzdin, A. A. (2014). Signaling pathway activation profiles make better markers of cancer than expression of individual genes. Oncotarget, 5.
Buzdin A A, Zhavoronkov A A, Korzinkin M B, Roumiantsev S A, Aliper A M, Venkova L S, Smirnov P Y and Borisov N M (2014) The OncoFinder algorithm for minimizing the errors introduced by the high-throughput methods of transcriptome analysis. Front. Mol. Biosci. 1:8. doi: 10.3389/fmolb 0.2014.00008, journal.frontiersin.org/Journal/10.3389/fmolb 0.2014.00008/full
Buzdin, A. A., Zhavoronkov, A. A., Korzinkin, M. B., Venkova, L. S., Zenin, A. A., Smirnov, Ph. Yu. and N. M. Borisov. OncoFinder, a new method for the analysis of intracellular signaling pathway activation using transcriptomic data. Frontiers in Genetics: Bioinformatics and Computational Biology, 2014, doi: 10.3389/fgene.2014.00055.
Conzelmann, H., Saez-Rodriguez, J., Sauter, T., Kholodenko, B. N., Gilles E. D. (2006). A domain-oriented approach to the reduction of combinatorial complexity in signal transduction networks. BMC Bioinformatics, 7, article 4. doi:10.1186/1471-2105-7-34.
Daniels, B. C., Chen, Y. J., Sethna, J. P., Gutenkunst, R. N., Myers, C. R. (2008). Sloppiness, robustness and evolvability in systems biology. Curr Opin Biotechnol, 19, 389-395. arXiv:0805.2628v1
Elkon, R., Vesterman, R., Amit, N. (2008). SPIKE—a database, visualization and analysis tool of cellular signaling pathways. BMC Bioinformatics, 9, article 110: doi: 10.1093/nar/gkq1167.
GEO repository URL: www.ncbi.nlm.nih.gov/geo/Haw,
Haw, R., Stein, L. (2012). Using the Reactome database. Curr Protoc Bioinformatics, June, chapter 8, unit 8.7. doi: 10.1002/0471250953.bi0807s38.
Kholodenko, B. N., Demin, O. V., Moehren, G., Hoek, J. B. (1999). Quantification of short term signaling by the epidermal growth factor receptor. J Biol Chem, 274, 30169-30181. doi: 10.1074/jbc.274.42.30169.
Kholodenko, B., Kiyatkin, A., Bruggeman, F., Sontag, E., et al. (2003). Untangling the wires: a strategy to trace functional interactions in signaling and gene networks, Proc Natl Acad Sci, 20, 12841-12846. doi:10.1073/pnas.192442699.
Kholodenko, B. N., Demin, O. V., Moehren, G, and. J. B. Hoek. (1999) Quantification of Short Term Signaling by the Epidermal Growth Factor Receptor. J. Biol. Chem, vol. 274, pp. 30169-81.
Kiyatkin, A., Aksamitiene, E., Markevich N. I., Borisov, N. M. et al. (2006). Scaffolding protein GAB1 sustains epidermal growth factor-induced mitogenic and survival signaling by multiple positive feedback loops. J Biol Chem, 281, 19925-19938. doi: 10.1074/jbc.M600482200.
Kuzmina, N. B., Borisov, N. M. Handling complex rule-based models of mitogenic cell signaling (On the example of ERK activation upon EGF stimulation). (2011). Intl Proc Chem Biol Envir Engng, 5, 76-82.
Mathivanan, S., Periaswamy, B., Gandhi, T., Kandasamy, K. et at. (2006). An evaluation of human protein-protein interaction data in the public domain. BMC Bioinformatics, 7, article S19. doi:10.1186/1471-2105-7-S5-S19.
Nakaya, A., Katayama, T., Itoh, M., Hiranuka, K. et al. (2013). KEGG OC: a large-scale automatic construction of taxonomy-based ortholog clusters. Nucleic Acids Res, January, 41. doi: 10.1093/nar/gks1239.
Nikitin, A., Egorov, S., Daraselia, N., Mazo, I. (2003). Pathway studio—the analysis and navigation of molecular networks. Bioinformatics, 19, 2155-2157. doi: 10.1093/bioinformatics/btg290.
SABiosciences, a Qiagen company. URL: www.sabiosciences.com/pathwaycentral.php
Ozerov, I. V., Lezhnina, K. V., Izumchenko, E., Artemov, A. V., Medintsev, S., Vanhaelen, Q., . . . & West, M. D. (2016). In silico Pathway Activation Network Decomposition Analysis (iPANDA) as a method for biomarker development. Nature Communications, 7, 13427.
The UniProt consortium. (2011). Ongoing and future developments at the Universal Protein Resource.Nucleic Acids Research, 39, D214-D219. doi: 10.1093/nar/gkq1020.
Yizhak K., Gabay O., Cohen H., Rupin E. (2013). Model-based identification of drug targets that revert disrupted metabolism and its application to ageing. Nature Communications, 4, 2632-doi: 10.1038/ncomms3632.
Zhavoronkov A, Buzdin A A, Garazha A V, Borissov N M and Moskalev A A (2014) Signaling pathway cloud regulation for in silico screening and ranking of the potential geroprotective drugs. Front. Genet. 5:49. doi: 10.3389/fgene.2014.00049.

What is claimed is:

1. A method for improving robustness of transcriptomic data analysis, the method comprising:
a. receiving cell transcriptomic data of a control group (C) and cell transcriptomic data (S) of group under study for a gene;
b. calculating a fold change ratio (fc) for said gene;
c. repeating steps a and b for a plurality of genes;
d. grouping co-expressed genes into modules;
e. estimating gene importance factors based on a network topology, mapped from a plurality of said modules; and
f. obtaining an insilico Pathway Activation Network Decomposition Analysis (iPANDA) value, said iPANDA value having a Pearson coefficient greater than a Pearson coefficient associated with another platform for manipulating said control cell transcriptomic data and said cell transcriptomic data of group under study for said plurality of genes.

2. A method according to claim 1, further comprising:
g. determining a biological an insilico Pathway Activation Network Decomposition Analysis (iPANDA) associated with at least one said module.

3. A method according to claim 2, further comprising:
h. providing a classifier for treatment response prediction of a drug to a disease, wherein said disease is selected from a cancer, a proliferative disease, an inflammatory disease and another disease or disorder.

4. A method according to claim 3, further comprising:
i. applying at least one statistical filtering test and a statistical threshold test to said fc values.

5. A method according to claim 4, further comprising:
j. obtaining proliferative bodily samples and healthy bodily samples from patients;
k. applying said drug to said patients; and
l. determining responder and non-responder patients to said drug.

6. A method according to claim 5, wherein said calculating step comprises comparing gene expression in at least one of selected signaling pathways and metabolic pathways.

7. A method according to claim 6, wherein said selected signaling pathways are associated with said drug.

8. A method according to claim 7, wherein said cancer is breast cancer.

9. A method according to claim 8, wherein said breast cancer is estrogen receptor negative (ERN) breast cancer.

10. A method according to claim 9, wherein said estrogen receptor negative (ERN) breast cancer is selected from HER2 positive and HER2 negative ERN breast cancer.

11. A method according to claim 10, wherein said drug is Paclitaxel.

12. A method according to claim 1, wherein said Pearson coefficient is greater than 0.7 and less than 1.

13. A method according to claim 12, wherein said Pearson coefficient is greater than 0.75 and less than 0.95.

14. A method according to claim 13, wherein said Pearson coefficient is greater than 0.75 and less than 0.95.

15. A method according to claim 13, wherein said Pearson coefficient is greater than 0.79 and less than 0.94.

16. A computer software product, said product configured for predicting drug efficacy for treating a disorder in a patient, the product comprising a computer-readable medium in which program instructions are stored, which instructions, when read by a computer, cause the computer to:
a. receive cell transcriptomic data of a control group (C) and cell transcriptomic data (S) of a group under study for a gene;
b. calculate a fold change ratio (fc) for said gene;
c. repeating steps a and b for a plurality of genes;
d. group co-expressed genes into modules;
e. estimate gene importance factors based on a network topology, mapped from a plurality of said modules; and
f. obtain an insilico Pathway Activation Network Decomposition Analysis (iPANDA) value, said iPANDA value having a Pearson coefficient greater than a Pearson coefficient associated with another platform for manipulating said control cell transcriptomic data and said cell transcriptomic data of group under study for said plurality of genes.

17. A system for predicting drug efficacy for treating a disorder in a patient the system comprising:
a. a processor adapted to activate a computer-readable medium in which program instructions are stored, which instructions, when read by a computer, cause the processor to:
i. receive cell transcriptomic data of a control group (C) and cell transcriptomic data (S) of group under study for a gene;
ii. calculate a fold change ratio (fc) for said gene;
iii. repeating steps a and b for a plurality of genes;
iv. group co-expressed genes into modules;
v. estimate gene importance factors based on a network topology, mapped from a plurality of said modules; and
vi. obtain an insilico Pathway Activation Network Decomposition Analysis (iPANDA) value, said iPANDA value having a Pearson coefficient greater than a Pearson coefficient associated with another platform for manipulating said control cell transcriptomic data and said cell transcriptomic data of a group under study for said plurality of genes.
b. a memory for storing said data; and
c. a display for displaying data associated with a predictive indication of said patient.
